(19) United States
(12) Plant Patent Application Publication
(10) Pub. No.: US 2016/0143198 P1
(43) Pub. Date: May 19, 2016

(54) Avocado tree named 'AO06'

(50) ;

(71) Applicant: HANS MERENSKY HOLDINGS (PTY) LTD, T/A WESTFALIA TECHNOLOGICAL SERVICES, Tzaneen (ZA)

(72) Inventor: Stefan Joseph Köhne, Tzaneen (ZA)

(73) Assignee: HANS MERENSKY HOLDINGS (PTY) LTD, T/A WESTFALIA TECHNOLOGICAL SERVICES

(21) Appl. No.: 14/544,018

(22) Filed: Nov. 14, 2014

(51) Int. Cl. A01H 5/00

(52) U.S. Cl. PLT/200

(57) ABSTRACT

The ‘AO.06’ avocado variety is notable for its large fruit size. The ‘AO.06’ variety is also flavorful and largely resistant to fungal attack.

### LATIN NAME OF THE GENUS AND SPECIES

Persea americana Mill.

### VARIETAL DENOMINATION

‘AO.06’

### BACKGROUND OF THE INVENTION

Avocado fruit size is one of the major issues that growers of most production areas face. The grower's net return can be doubled when prices from small fruit and big fruit are compared.

Currently, the main avocado variety grown in the world is ‘Hass’ (U.S. Plant Pat. No. 139). Many other avocado varieties have been created and patented in the last twenty years, but none of them has been able to obtain important interest from growers. There are several reasons for the lack of interest in these other avocado varieties, including flavor or taste, size, and appearance of the other varieties.

### SUMMARY OF THE INVENTION

‘AO.06’ is a new and distinct variety of avocado tree Persea americana Mill. The ‘AO.06’ variety was discovered in a cultivated avocado orchard located near Tzaneen, South Africa. The discovered tree had fruit size which was significantly larger than the avocado fruit from other avocado trees, including ‘Hass’. After evaluations, it was determined that the ‘AO.06’ tree consistently produced larger fruit than other avocado varieties. It was also found that the avocado fruit of the ‘AO.06’ has a desirable flavor and is largely resistant to fungal attack.

### BRIEF DESCRIPTION OF THE DRAWINGS

The accompanying photographs show the specimens of the tree and plant parts of the ‘AO.06’ variety.

FIG. 1 is a photograph of an ‘AO.06’ tree;

FIG. 2 is a photograph depicting leaves of the ‘AO.06’ variety;

FIG. 3 is a photograph depicting a front side of a single leaf of the ‘AO.06’ variety;

FIG. 4 is a photograph depicting the back side of a leaf of the ‘AO.06’ variety;

FIG. 5 is a photograph depicting an ‘AO.06’ tree flowering;

FIG. 6 is a photograph depicting flower buds of the ‘AO.06’ variety;

FIG. 7 is a photograph depicting fruit of the ‘AO.06’ variety;

FIG. 8 is a photograph depicting fruit of the ‘AO.06’ variety, with a hand placed thereunder for size comparison purposes;

FIG. 9 is a photograph depicting an immature fruit to the right of a mature fruit of the ‘AO.06’ variety;

FIG. 10 is a photograph depicting a mature ‘Edranol’ avocado fruit to the right of a mature ‘AO.06’ variety fruit; and

FIG. 11 is a photograph depicting a sectioned fruit, illustrating the fruit flesh and stone, of the ‘AO.06’ variety.

### DETAILED BOTANICAL DESCRIPTION OF THE VARIETY

The following is a detailed botanical description of the ‘AO.06’ variety. This description is based on observation of trees which were asexually reproduced from the mother tree. The mother tree was discovered in a cultivated area near Tzaneen, South Africa. A multi-year study of the variety has concluded that the characteristics of the plant are stable.

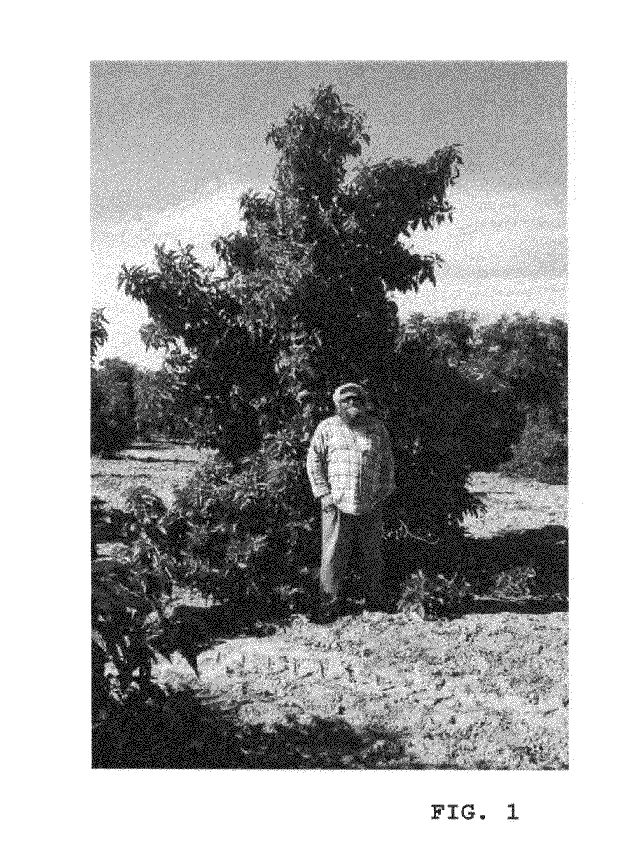

FIG. 1 is a photograph depicting a mature ‘AO.06’ variety tree. The tree has a growth habit which is semi-drooping.

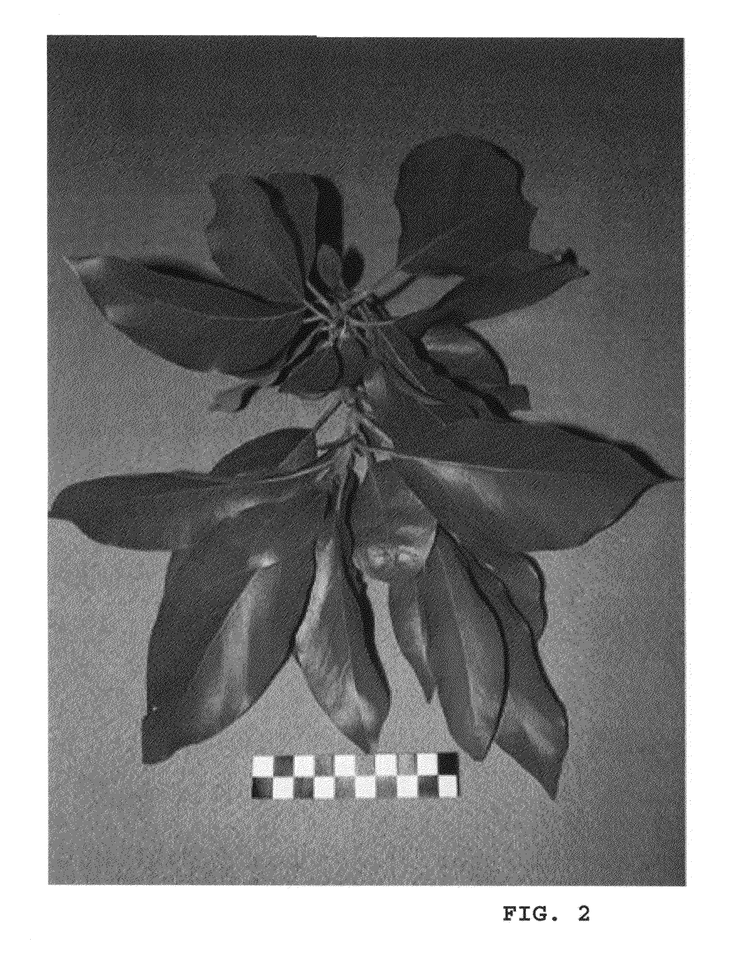

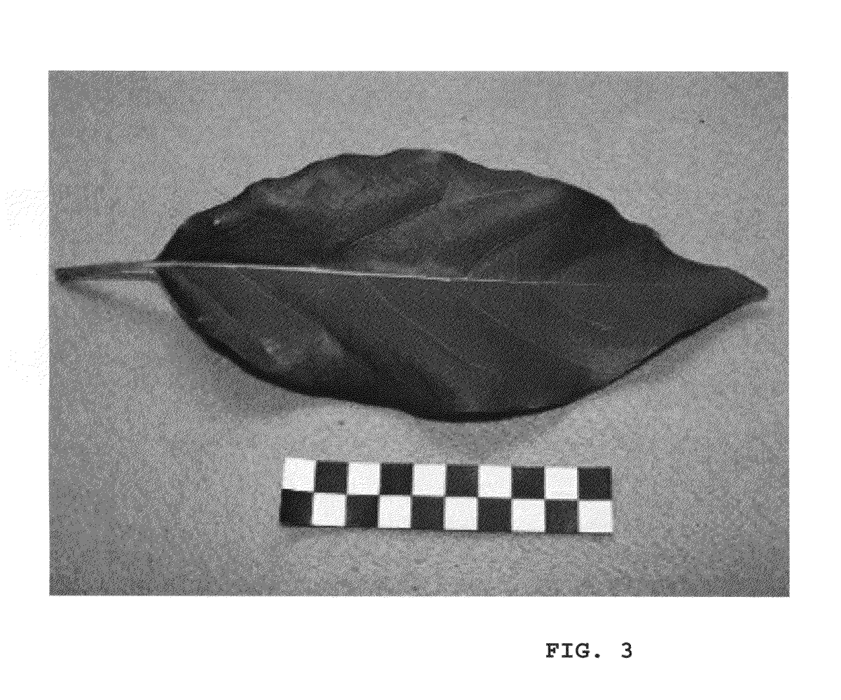

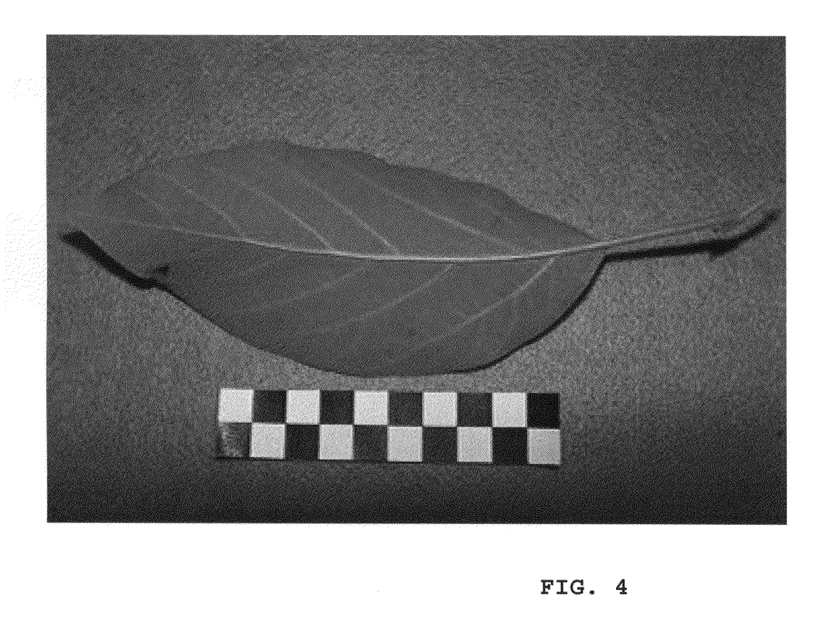

FIGS. 2-4 are photographs depicting the leaves of the ‘AO.06’ variety. The young shoots have a reddish color. The lenticels of the young shoots are a green color. The pubescence of the petiole of the young leaf is a white color.

The attitude of the leaf relative to the shoot is outwards. The leaf blade is of a medium length and width. The ratio of the length/width of the leaf blade is also medium.

The shape of the leaf blade is lanceolate to elliptic. The shape of the apex of the leaf blade is acuminate. Twisting along the whole length of the leaf blade is absent. However, twisting of the apex of the leaf blade is present, with a very slight twisting at the apex of the leaf blade. Undulation at the margin of the leaf blade is absent or very weak.

Relief of venation on the upper surface of the leaf blade is level. The number of secondary veins of the leaf blade is intermediate. The density of pubescence on the lower surface of the leaf blade is absent or sparse. Moreover, anise aroma of the leaf blade is absent.

The length of the petiole is medium. The length of the axis of inflorescence is short to medium. The color of lenticels of the inflorescence is green. Pubescence of the inner surface of the sepal is present. However, the density of pubescence on the inner surface of the sepal is sparse.

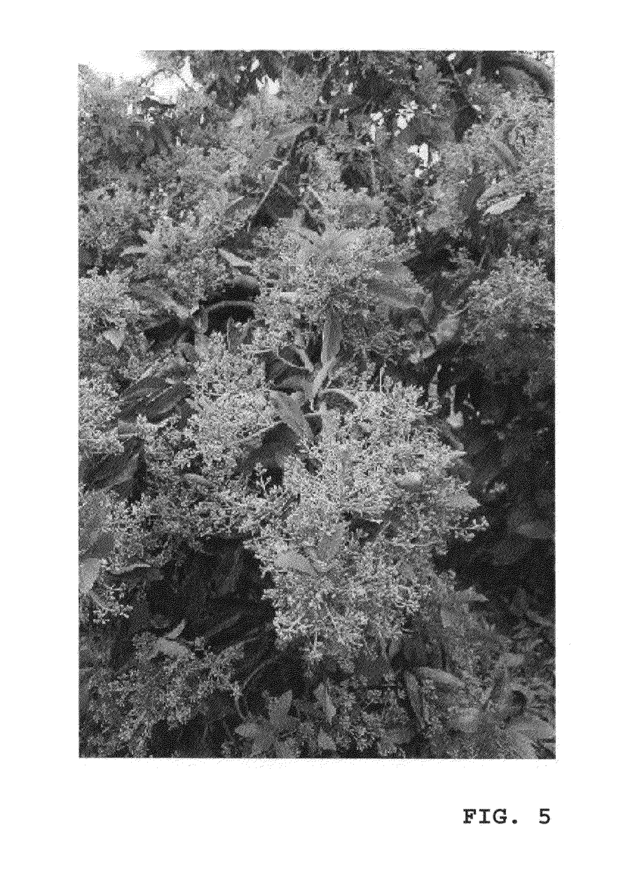

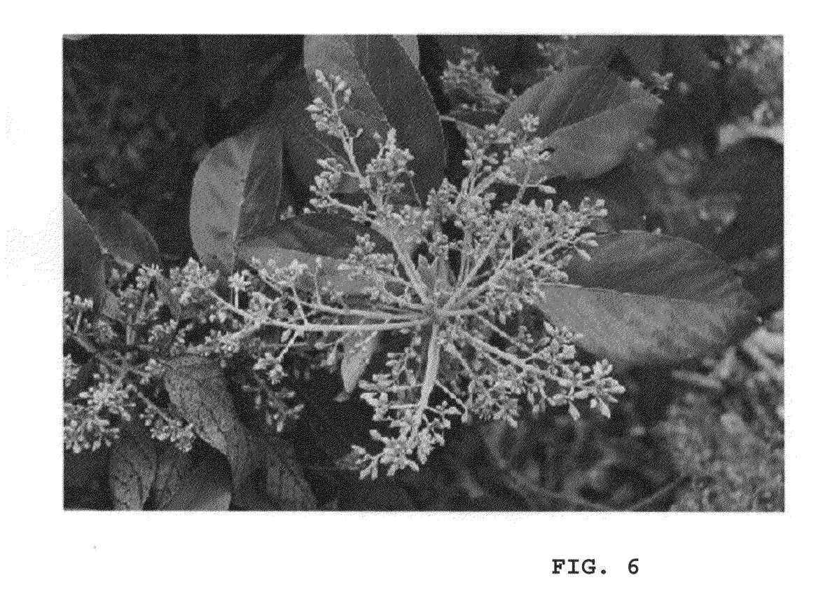

FIGS. 5 and 6 illustrate blooms of the ‘AO.06’ variety. The time of beginning of flowering has been found to be early to medium.

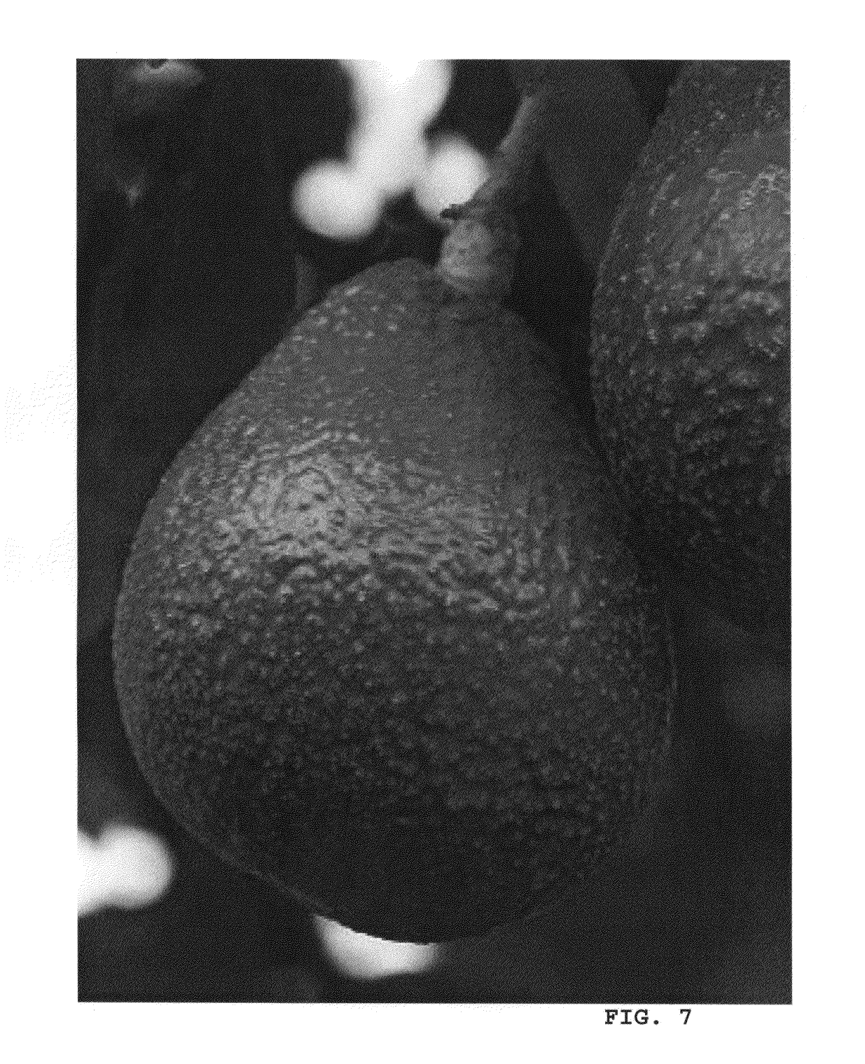

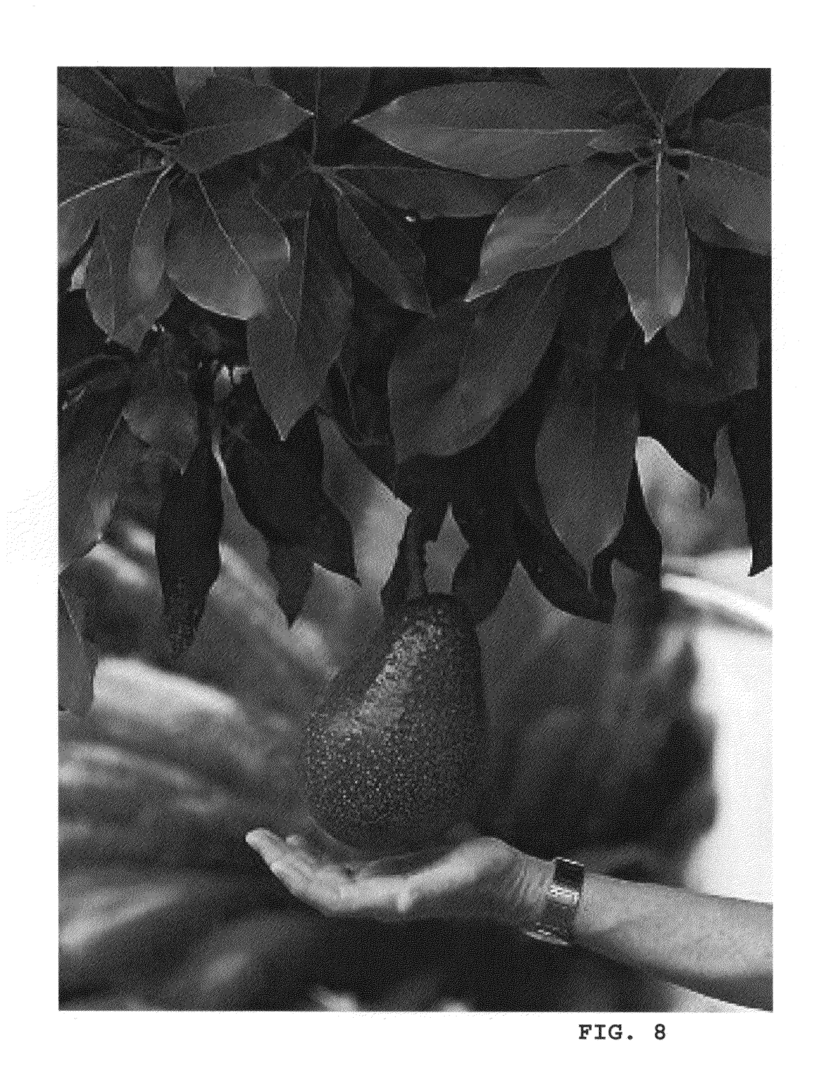

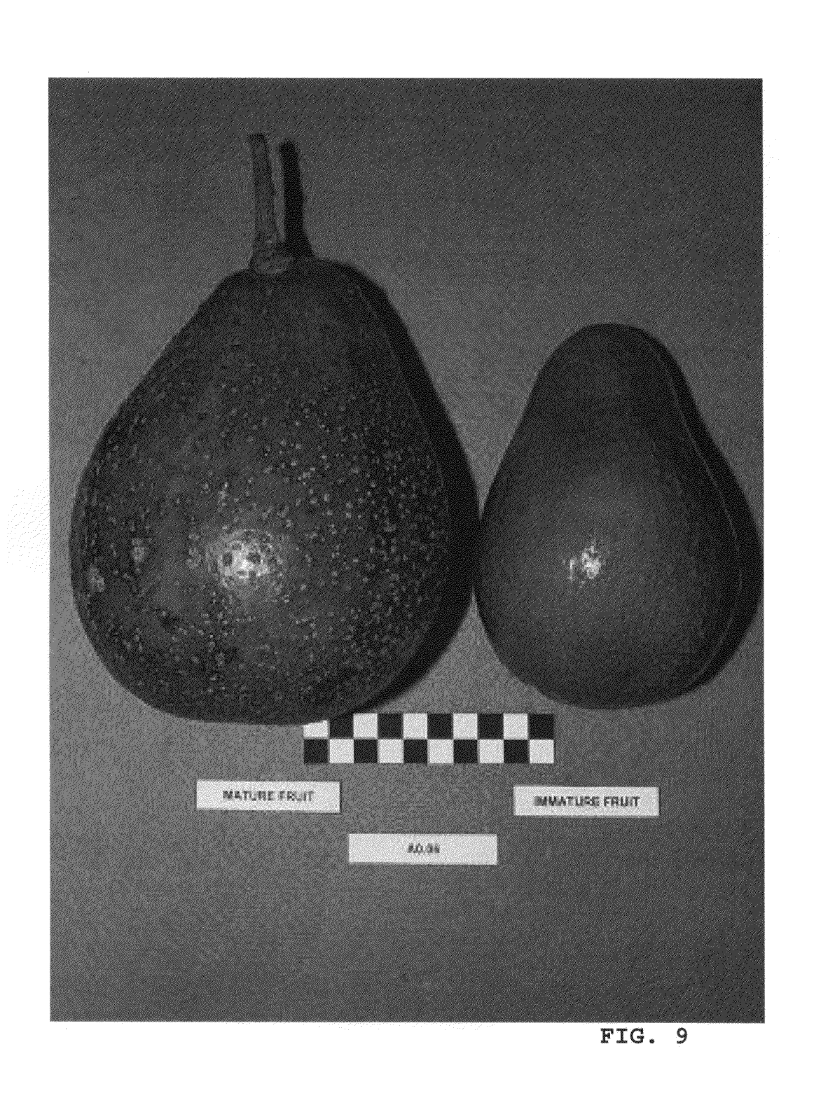

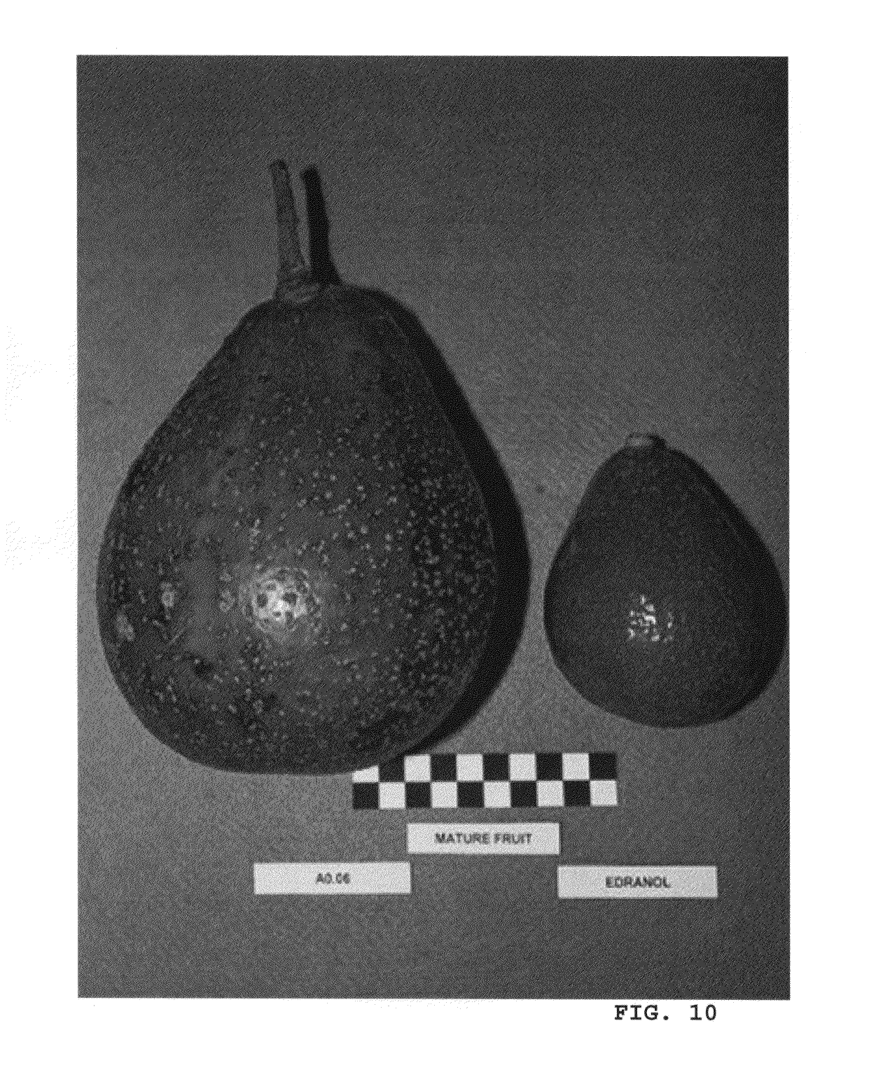

FIGS. 7-10 illustrate the fruit of the ‘AO.06’ variety. A primary distinguishing feature of the ‘AO.06’ variety is that it has much larger mature fruit than other avocado varieties. This is particularly shown in FIG. 8, with the mature fruit hanging on the tree of the ‘AO.06’ variety, with a man's hand placed thereunder. FIG. 10 illustrates the mature fruit of the ‘AO.06’ variety, as compared to the mature fruit of the ‘Edranol’ variety, illustrating that the ‘AO.06’ variety fruit is several times larger than the mature fruit of ‘Edranol’.

The length of the mature fruit is long. The diameter of the mature fruit is medium. The ratio of the length/width of the mature fruit is small to medium.

The shape of the stalk end of the mature fruit is pointed to narrowly rounded. A neck is present in the mature fruit. A depression is also present at the stalk end of the mature fruit. The diameter of the stalk attachment of the mature fruit is medium. The stalk is slightly oblique in position in the mature fruit. The stylar region of the mature fruit is flattened in shape.

The conspicuousness of lenticels is medium to strong in the mature fruit. The lenticels are of a medium size in the mature fruit. The lenticels of the mature fruit are brown in color.

The pedicel is thicker compared to the peduncle, at the junction thereof. The pedicel is short to medium in length and has a conical shape. A “nailhead” is absent in the pedicel. The pedicel has a yellow-green color. The pedicel surface is wrinkled.

The mature fruit has a medium to rough surface, having a medium to strong glossiness. The color of the ripe fruit is medium green. The thickness of the skin of the ripe fruit is medium. The ripe fruit has a corky skin consistency.

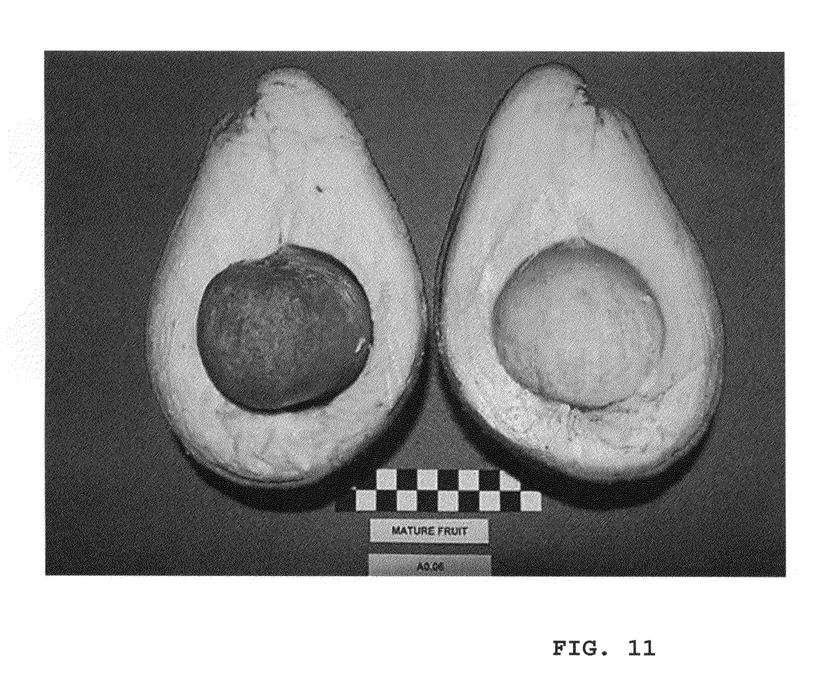

FIG. 11 illustrates a fruit of the ‘AO.06’ variety sectioned so as to illustrate the flesh and seed or stone thereof. The adherence of the skin to the flesh in the ripe fruit is weak. The main color of the flesh of the ripe fruit is yellow (RHS yellow 10C, 11C). The color of the layer of the ripe fruit next to the skin is a light to medium green. The width of the layer next to the skin of the ripe fruit is medium.

Fibers in the flesh of the ripe fruit are inconspicuous. The consistency of the flesh of the ripe fruit is buttery. Anise aroma is absent in the ripe fruit. The ratio of the fruit length/seed length of the ripe fruit is medium.

The shape of the seed in longitudinal section (lateral view) is depressed oblate. The shape of the seed in cross-section is circular. Adherence of the flesh to the seed coat is absent or weak. The cotyledon surface is wrinkled. The adherence of the seed coat to the cotyledon is absent or weak. The seed coat surface is smooth or slightly wrinkled.

As indicated above, a particular unique characteristic of the ‘AO.06’ variety is that the fruit is very large. It has also been found to be very flavorful and good eating. The variety has also been found to be largely resistant to fungal and even insect attack.

What is claimed is:

1. A new and distinct variety of avocado tree, substantially as illustrated and described herein.
